(19) United States
(12) Patent Application Publication
(10) Pub. No.: US 2003/0199421 A1
(43) Pub. Date: Oct. 23, 2003

(54) Scented plastic tag

(76) Inventor: Debra K. Copfer, Salt Lake City, UT (US)

Correspondence Address: Debra K. Copfer, 2183 E Wilson Ave, Salt Lake City, UT, 84108, US

(21) Appl. No.: 10/127,738

(22) Filed: Apr. 22, 2002

(51) Int. Cl. A61K 7/46

(52) U.S. Cl. 512/1; 512/4

(57) ABSTRACT

A plastic tag (100) releasing a pleasant fragrance, scent or aroma used for pets, luggage, purse, key chains or other belongings made of a polymer mix impregnated with a fragrance and attached to the desired article with a through-hole (300) using a metal fastener (400), and can be used as an air freshener.

### CROSS-REFERENCED TO RELATED APPLICATIONS

| | | | |
|---|---|---|---|
| 195324 | September 1877 | Atkinson. | 239/6. |
| 3567119 | March 1971 | Wilbert et al. | 63/2. |
| 4043858 | August 1977 | Dantowitz. | 156/251. |
| 4112603 | September 1978 | Giulie. | 40/638. |
| 4215497 | August 1980 | Levy. | 40/649. |
| 5040394 | August 1991 | Wang. | 70/457. |
| 5429393 | July 1995 | Parlo. | 283/75. |
| 5690365 | November 1997 | Fascenelli, Jr. et al. | 283/67. |
| 6051547 | April 2000 | Omitz. | 512/1. |
| 6089947 | July 2000 | Green. | 446/268. |
| 6235705 | May 2001 | Zembrodt et al. | 510/519. |
| 6357260 | March 2002 | Lutz. | 63/1.11 |

### BACKGROUND

1. Field of Invention

The present invention relates generally to the field of tags for pets, luggage, key chains or other belongings and most particularly to a new and useful tag containing a fragrance

2. Description of the Prior Art

Pets are traditionally known for their unpleasant odor in just a days after being bathed. Their owners will use fragrance sprays on their pets to mask the malodor. Such sprays come in variety of scents. These sprays are often selected for their appealing fragrance and aroma. Each fragrance has its own special quality, which makes it particularly attractive for the consumer; however, the fragrance of a perfume or cologne quickly evaporates with the passage of time or is washed away when the pet takes a bath. In addition, some pets cannot wear particular fragrances because they are sensitive to its contents. As a result, some fragrances cannot be worn at all by those pets.

It is further known that most tags known in the art are for identification purposes only and carry no fragrance. Consequently, if the consumer desires to carry the preferred fragrance with them in an easily attachable compact manner such as inside their purse, on a key chain or other belongings they are limited only to the large bulky designs that must be tucked away inside their cars such as under their seat, or left sitting on the end table in the house. The consumer enjoys having the pleasant scent emitting from the current freshener; however, they often have major drawbacks to their use, such as the large size makes it undesirable to their carry it in their purse, on a key chain or other belongings.

Disclosed in U.S. Pat. No. 4,215,497 a heart-shaped tag has two plastic pieces secured together with the identification member sandwiched between the plastic pieces. No fragrance is associated with this tag.

A kit for making identification tags for pets and other belongings is disclosed in U.S. Pat. No. 5,690,365. This kit contains a method of applying the pet identification information on the tag base, and then baking a shrinkable plastic. The process produces a hard plastic identification tag. This tag has no fragrance associated with it.

U.S. Pat. No. 195,324 discloses a scented charm for use on a bracelet or necklace. The charm is made of porous earthenware and scented by exposing the charm to a liquid perfume for absorption by the earthenware.

U.S. Pat. No. 3,567,119 teaches a method of improving a diffusion of fragrances from polymers. Example 1 of the patent discloses a method of making perfume-impregnated polyethylene beads by adding an anionic surfactant to the perfume in the example is about 3% by weight.

U.S. Pat. No. 6,357,260 teaches a method of making a scented friendship bracelet kit for children, which contains several ornamental beads strung on a string. In addition a mesh bag holds a plurality of polymer pellets impregnated with a fragrance.

### SUMARY

In accordance with the present invention a plastic tag comprises a pleasant fragrance impregnated in a polymer mix with the slow sustained release of a fragrant molecule being a desirable trait. The fragrance will remain for a long period of time, such as 30 days.

Objects and Advantages

It is an object of the present invention to provide a plastic tag attached to the collar of a pet, fastened to piece of luggage or a purse, used on a key chain or attached to other belongings, which will provide a pleasant fragrance, scent or aroma.

It is an object of the invention to provide a fragranced tag, which can be produced in a variety of colors, shapes and sizes.

It is an object of the invention to provide a fragrance tag, which the fragrance will last for a long period of time, such as 30 days.

It is further an object that the invention can be used as an air freshener.

### BRIEF DESCRIPTION OF DRAWINGS

In the drawings:

FIG. 1 is a perspective front view of the tag with attachment device; and

FIG. 2 is a perspective view of the tag in a side view.

### REFERENCE NUMERALS IN DRAWINGS

100 tag with front view

200 tag side view

300 through-hole

400 metal fastener

### DESCRIPTION OF THE PREFERRED EMBODIMENTS

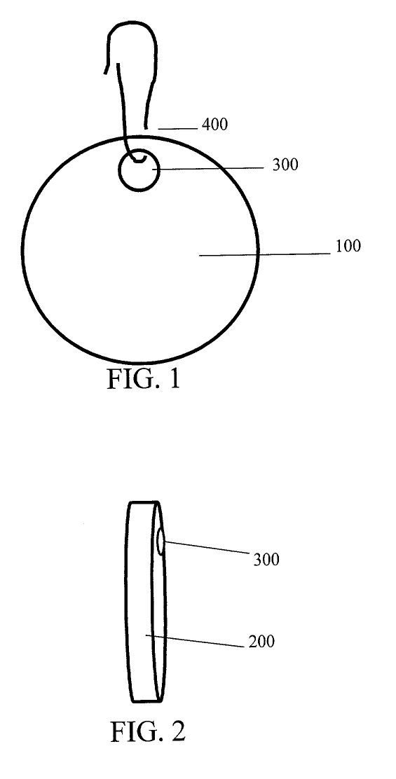

Referring now to the drawings, in which like reference numerals are used to refer to the same or similar elements. FIG. 1 shows a basic view of the front of the tag 100. The tag 100 may come in a variety of shapes and sizes and each will have a through-hole 300 for attaching the metal fastener 400. The amount of fragrance diffused by the carrier will be increased the greater the surface area of the carrier.

FIG. 2 shows the tag 100 in a side view 200. The tag is made of preferably a polyethylene, polypropylene or mixtures of the two, impregnated with about 25% or more by weight fragrance, which is suitable for use with the selected polymer. The fragranced polymer will continuously provide a scent over a length of time, such as 30 days.

As an alternative to using the polymer tag 100 attached to the designated article, the tag 100 can be placed in a drawer as a sachet or as an air freshener.

Thus, there is shown and described a unique design and concept of uses of this tag. While this description is directed to a particular embodiment, it is understood that those skilled in the art may conceive modification and/or variations to the specific embodiments shown and described herein. Any such modifications or variations which fall within the purview of this description herein is intended to be illustrative only and is not intended to be limitative. Rather, the scope of the invention described herein is limited only by the claims appended hereto.

What is claimed is:

1. A plastic tag comprising a fragrance carrier means for containing a fragrance and diffusing the fragrance.

2. The tag recited in claim 1, wherein the tag is made from a polyethylene, polypropylene and mixtures thereof.

3. The tag recited in claim 2, wherein the fragrance is impregnated in a mixture of polyethylene, polypropylene and mixtures thereof.

4. The tag recited in claim 3, wherein the fragranced polyethylene, polypropylene and mixtures thereof are impregnated with about 25% or more by weight of fragrance.

5. The tag recited in claim 4, wherein the tag may be made in a variety of shapes and sizes.

6. The tag recited in claim 5, wherein attachment means comprises a metal fastener.
